(19) United States
(12) Patent Application Publication
(10) Pub. No.: US 2025/0102552 A1
(43) Pub. Date: Mar. 27, 2025

(54) METHOD, APPARATUS AND SYSTEM FOR EVALUATING PERFORMANCE OF ELECTROMAGNETIC RADIATION DEVICE

(71) Applicant: ELECTRONICS AND TELECOMMUNICATIONS RESEARCH INSTITUTE, Daejeon (KR)

(72) Inventors: Kwang Jae LEE, Daejeon (KR); Won Young SONG, Sejong-si (KR); Soon Ik JEON, Daejeon (KR)

(21) Appl. No.: 18/787,916

(22) Filed: Jul. 29, 2024

(30) Foreign Application Priority Data
Sep. 27, 2023 (KR) 10-2023-0130917

(51) Int. Cl. G01R 29/08

(52) U.S. Cl. CPC G01R 29/0885; G01R 29/0821; G01R 29/0892

(57) ABSTRACT

A method, an apparatus and a system for evaluating the performance of an electromagnetic radiation device are disclosed. According to an embodiment of a present disclosure, a performance evaluation system includes a power source, an impedance matching circuit, the electromagnetic radiation electrode and a phantom that simulates a subject which is a target of electromagnetic wave irradiation. The performance evaluation system also includes an instrument for measuring the power of the power source, a first power inside the phantom, and a first electric field inside the phantom. The performance evaluation system also includes an analysis device for calculating a power radiated to the phantom and evaluating performance of an electromagnetic radiation device using at least one of the power radiated to the phantom, a second power inside the phantom, and a second electric field inside the phantom.

### CROSS-REFERENCE TO RELATED APPLICATIONS

The present application is based on, and claims priority from, Korean Patent Application Number 10-2023-0130917, filed Sep. 27, 2023, the disclosure of which is incorporated by reference herein in its entirety.

### TECHNICAL FIELD

The present disclosure relates to a method, an apparatus and a system for evaluating the performance of an electromagnetic radiation device. More specifically, the present disclosure relates to a method and system for evaluating the performance of an electromagnetic radiation device by acquiring measurement data using a phantom, generating adjustment data by applying an electromagnetic correction algorithm to the measurement data, and graphing the adjustment data.

### BACKGROUND

The contents described below merely provide background information related to the present disclosure and do not constitute prior art.

An electromagnetic radiation device is a device that freely controls electromagnetic waves with penetrating power to be radiated to a subject. The electromagnetic radiation device is used in various fields such as communications, home appliances, transportation, or medical fields. In the medical field, the electromagnetic radiation device is used to diagnose or treat diseases by irradiating electromagnetic waves into the human body.

Since the internal structure of the human body is structurally complex, the precision of electromagnetic wave irradiation has a significant impact on the reliability of diagnosis and treatment results. Accordingly, it is necessary to evaluate whether a medical electromagnetic radiation device operates exactly as designed to achieve the purpose of diagnosing and treating diseases. In addition, in order to achieve this purpose, it is necessary to check the operating state of the medical electromagnetic radiation device and monitor it in real time.

### SUMMARY

In view of the above, the present disclosure provides a method, an apparatus and a system for evaluating the performance of an electromagnetic radiation device using a phantom.

Further, according to one embodiment, the present disclosure provides a method, an apparatus and a system for evaluating the performance of an electromagnetic radiation device by correcting data measured from a phantom to generate adjustment data and graphing the adjustment data.

In addition, according to one embodiment, the present disclosure provides a system that monitors the operating state of an electromagnetic radiation device in real time.

The objects to be achieved by the present disclosure are not limited to the objects mentioned above, and other objects not mentioned will be clearly understood by one of ordinary skill in the art from the description below.

According to the present disclosure, a performance evaluation system includes a power source for transmitting power to an electromagnetic radiation electrode. The performance evaluation system also includes an impedance matching circuit for matching an impedance of the power source and an impedance of the electromagnetic radiation electrode. The performance evaluation system also includes the electromagnetic radiation electrode for irradiating a phantom with electromagnetic waves using power of the power source. The performance evaluation system also includes the phantom that simulates a subject which is a target of electromagnetic wave irradiation. The performance evaluation system also includes an instrument for measuring the power of the power source, a first power inside the phantom, and a first electric field inside the phantom. The performance evaluation system also includes an analysis device for calculating a power radiated to the phantom and evaluating performance of an electromagnetic radiation device using at least one of the power radiated to the phantom, a second power inside the phantom, and a second electric field inside the phantom.

According to the present disclosure, a monitoring system includes a power source for transmitting power to an electromagnetic radiation electrode. The monitoring system includes an impedance matching circuit for matching an impedance of the power source and an impedance of the electromagnetic radiation electrode. The monitoring system includes the electromagnetic radiation electrode for irradiating a phantom with electromagnetic waves using power of the power source. The monitoring system includes the phantom that replaces a subject which is a target of electromagnetic wave irradiation. The monitoring system includes a detection unit configured to detect a power difference between a power signal of the power source and a measurement signal inside the phantom and a phase difference between the power signal of the power source and the measurement signal inside the phantom. The monitoring system includes an analysis device for monitoring an operating state of an electromagnetic radiation device using at least one of the power difference and the phase difference.

According to the present disclosure, a method performed by a performance evaluation system includes transmitting power of a power source to an electromagnetic radiation electrode. The method also includes irradiating a phantom with electromagnetic waves using the power of the power source. The method also includes measuring the power of the power source, a first power inside the phantom, and a first electric field inside the phantom. The method also includes calculating power radiated to the phantom, a second power inside the phantom, and a second electric field inside the phantom. The method also includes evaluating performance of an electromagnetic radiation device using at least one of the power radiated to the phantom, the second power inside the phantom, and the second electric field inside the phantom.

According to the present disclosure, it is possible to increase the reliability of electromagnetic wave irradiation results by evaluating the performance of the electromagnetic radiation device using a phantom.

According to one embodiment of the present disclosure, it is possible to predict the performance of the electromagnetic wave irradiation results and ensure safety by correcting the data measured from the phantom to generate adjustment data and graphing the adjustment data to evaluate the performance of the electromagnetic radiation device.

According to one embodiment of the present disclosure, it is possible to check the operating state of the electromagnetic radiation device in real time using the system that monitors the operating state of the electromagnetic radiation device in real time.

The effects of the present disclosure are not limited to the effects mentioned above, and other effects not mentioned will be clearly understood by one of ordinary skill in the art from the following description.

### BRIEF DESCRIPTION OF THE DRAWINGS

FIG. 1 is a structural diagram for explaining an electromagnetic radiation device according to one embodiment of the present disclosure.

FIG. 2 is a structural diagram for explaining a phantom according to one embodiment of the present disclosure.

FIG. 3 is a structural diagram for explaining a phantom according to another embodiment of the present disclosure.

FIG. 4 is a structural diagram for explaining a system for evaluating the performance of the electromagnetic radiation device, according to one embodiment of the present disclosure.

FIG. 5 is a flowchart for explaining a process of generating reference data and adjustment data according to one embodiment of the present disclosure.

FIG. 6 is a structural diagram for explaining a system for monitoring the operating state of the electromagnetic radiation device, according to another embodiment of the present disclosure.

FIGS. 7A and 7B are structural diagrams for explaining a detection unit and a comparator included in the detection unit, according to one embodiment of the present disclosure.

FIG. 8 is a flowchart for explaining a method of evaluating the performance of the electromagnetic radiation device, according to one embodiment of the present disclosure.

### DETAILED DESCRIPTION

Hereinafter, some exemplary embodiments of the present disclosure will be described in detail with reference to the accompanying drawings. In the following description, like reference numerals preferably designate like elements, although the elements are shown in different drawings. Further, in the following description of some embodiments, a detailed description of known functions and configurations incorporated therein will be omitted for the purpose of clarity and for brevity.

Additionally, various terms such as first, second, A, B, (a), (b), etc., are used solely to differentiate one component from the other but not to imply or suggest the substances, order, or sequence of the components. Throughout this specification, when a part ‘includes’ or ‘comprises’ a component, the part is meant to further include other components, not to exclude thereof unless specifically stated to the contrary. The terms such as ‘unit’, ‘module’, and the like refer to one or more units for processing at least one function or operation, which may be implemented by hardware, software, or a combination thereof.

The following detailed description, together with the accompanying drawings, is intended to describe exemplary embodiments of the present disclosure, and is not intended to represent the only embodiments in which the present disclosure may be practiced.

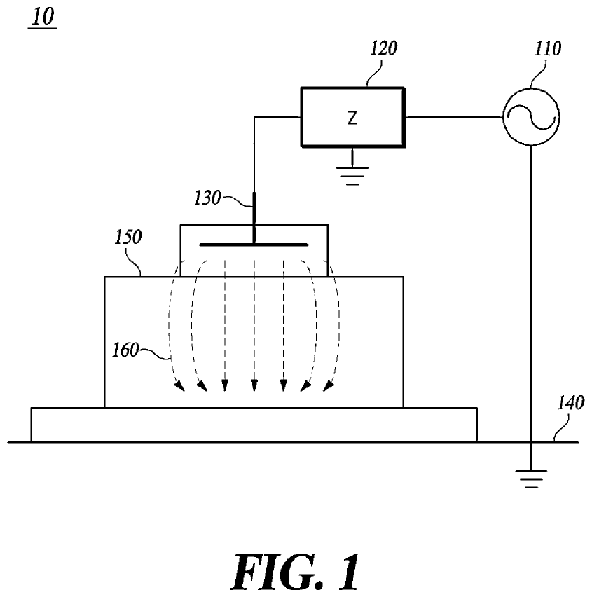

FIG. 1 is a structural diagram for explaining an electromagnetic radiation device according to one embodiment of the present disclosure.

Referring to FIG. 1, an electromagnetic radiation device 10 includes all or part of a power source 110, an impedance matching circuit 120, an electromagnetic radiation electrode 130, and a counter electrode 140. The power source 110 may generate power and transmit it to the electromagnetic radiation electrode 130 through the impedance matching circuit 120. The impedance matching circuit 120 may match the impedance of the power source 110 and the impedance of the electromagnetic radiation electrode 130. In this case, the impedance matching is performed to correct the difference in impedance between the two connection ends to minimize return loss due to the difference in impedance. Accordingly, maximum power can be transmitted to the electromagnetic radiation electrode 130.

The electromagnetic radiation electrode 130 may irradiate a subject 150 with electromagnetic waves using power received from the power source 110. As the electromagnetic radiation electrode 130 irradiates the subject 150 with electromagnetic waves, an electromagnetic field 160 may be formed inside the subject 150. The electromagnetic radiation electrode 130 may be connected to a (+) electrode and the counter electrode 140 may be connected to a (−) electrode. As another example, when the electromagnetic wave irradiation electrode 130 is connected to the (−) electrode, the counter electrode 140 may be connected to the (+) electrode.

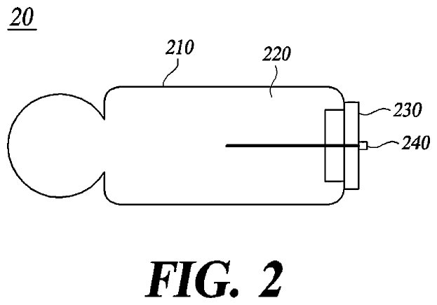

FIG. 2 is a structural diagram for explaining a phantom according to one embodiment of the present disclosure.

Referring to FIG. 2, a phantom 20 is a tool for evaluating the performance of the electromagnetic radiation device 10. Instead of a subject, the phantom 20 becomes the target of electromagnetic wave irradiation by the electromagnetic radiation device 10. The phantom 20 includes all or part of a plastic container 210, a biological mimicry fluid 220, an opening and closing lid 230, and an input/output sensor terminal 240. The plastic container 210 contains a biological mimicry fluid 220. The plastic container 210 can prevent leakage of the biological mimicry fluid 220.

The biological mimicry fluid 220 is a solution that is in liquid form and mimics the electrical characteristics of a living body. In another embodiment, the biological mimicry fluid 220 may be in a semisolid form. In this case, agar or gelatin may be used as a raw material for the biological mimicry fluid 220. There may be electric field sensors for measuring electric field intensity and power inside the biological mimicry fluid 220. The electric field sensors may measure the electric field intensity and power at a plurality of points inside the biological mimicry fluid 220 while rotating. The electric field sensors may be placed inside the biological mimicry fluid 220 in various forms.

The opening and closing lid 230 is used to open and close the plastic container 210. The opening/closing lid 230 may be removed from the plastic container 210 in order to take out the biological mimicry fluid 220 from the plastic container 210 and store it separately. The input/output sensor terminal 240 may transmit information about electric field strength and power measured by the electric field sensors to an external instrument.

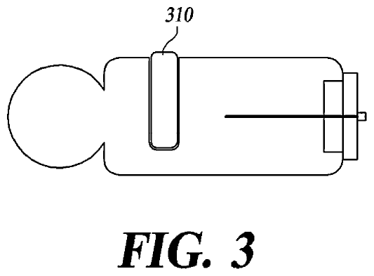

FIG. 3 is a structural diagram for explaining a phantom according to another embodiment of the present disclosure.

Referring to FIG. 3, when the phantom is irradiated with electromagnetic waves and the temperature of the phantom increases, the electric field sensors inside the biological mimicry fluid 220 cannot measure the temperature. Accordingly, a biomimetic solid specimen 310 may be added to the structure of the phantom 20 of FIG. 2 to measure temperature. The biomimetic solid specimen 310 is a solid specimen that simulates the electrical characteristics of a living body. A slot may be formed in the plastic container 210 of the phantom 20 to insert the biomimetic solid specimen 310. The biomimetic solid specimen 310 may be inserted into the slot. The biomimetic solid specimen 310 may be present inside another plastic container or alternative packaging structure. Temperature sensors may be present inside the biomimetic solid specimen 310. The temperature sensors may measure the temperature inside the phantom when electromagnetic waves are irradiated.

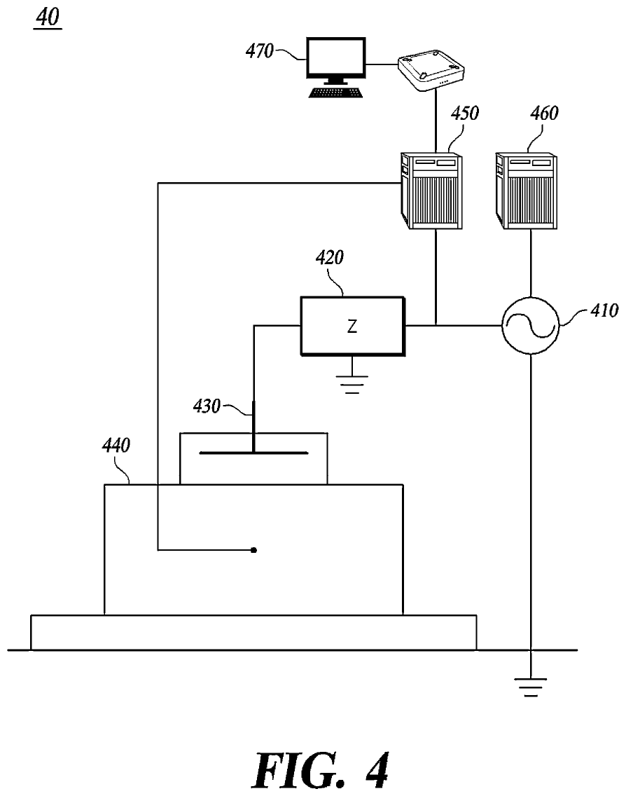

FIG. 4 is a structural diagram for explaining a system for evaluating the performance of the electromagnetic radiation device, according one an embodiment of the present disclosure.

Referring to FIG. 4, the system for evaluating the performance of the electromagnetic radiation device (hereinafter referred to as ‘performance evaluation system’) 40 includes a power source 410, an impedance matching circuit 420, an electromagnetic radiation electrode 430, a phantom 440, a power meter 450, a power source meter 460, and an analysis device 470. The power source 410 may transmit power to the electromagnetic radiation electrode 430 through the impedance matching circuit 420. The electromagnetic radiation electrode 430 may irradiate the phantom 440 with electromagnetic waves using power received from the power source 410.

When the phantom 440 is irradiated with electromagnetic waves, the electromagnetic waves can be converted into heat inside the phantom 440. When the phantom 440 is irradiated with electromagnetic waves, an electromagnetic field is formed, and the electric field can be converted into power loss density (PLD) based on the conductivity of the phantom 440. PLD is the power consumed per unit volume. PLD can be expressed as Equation 1.

$$P L D = \frac{σ {\vert E \vert}^{2}}{2} [ W / {m}^{3} ] \qquad ( Equation 1 )$$

Here, σ is the conductivity (S/m) of the phantom 440, and E is the electric field (V/m) inside the phantom 440. Power consumed from inside the phantom 440 may be converted into heat. Accordingly, the PLD calculated from inside the phantom 440 may be a heat source. The PLD continuously increases the temperature inside the phantom 440. The temperature change due to PLD, ΔT, can be expressed as Equation 2.

$$Δ T = \frac{PLD}{c \cdot ρ} Δ t \qquad ( Equation 2 )$$

Here, Δt is the time change, ρ is the density inside the phantom 440, and c is the specific heat capacity. The power meter 450 may be a vector network analyzer. The vector network analyzer is a device that measures electrical network parameters. The power meter 450 may measure the electric field inside the phantom 440 using the electric field sensor of the phantom 440. The analysis device 470 may calculate the PLD using the conductivity of the phantom 440 and the measured electric field.

The power source meter 460 may measure the power output from the power source 410. The power meter 450 may measure the power inside the phantom 440 using the electric field sensor of the phantom 440. The analysis device 470 may calculate a relative power ratio of the phantom 440 using the power output from the power source 410 and the power inside the phantom 440.

The analysis device 470 may predict the pattern and distribution of power inside the phantom 440 using the relative power ratio and PLD of the phantom 440. The analysis device 470 may calculate the power radiated to the phantom 440 using the relative power ratio of the phantom 440 and the PLD. The analysis device 470 may evaluate the performance of the electromagnetic radiation device using the power radiated to the phantom 440, the power inside the phantom 440, and the electric field inside the phantom 440.

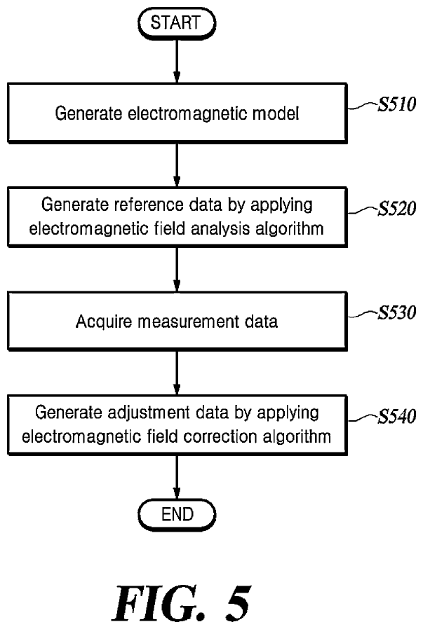

FIG. 5 is a flowchart for explaining a process of generating reference data and adjustment data according to an embodiment of the present disclosure.

Referring to FIG. 5, the analysis device 470 may generate an electromagnetic model (S510). The analysis device 470 may generate the electromagnetic model using information about the phantom 440, information about the electromagnetic radiation electrode 430, information about the counter electrode, information about the operating frequency, and information about the impedance matching circuit 420. The information about the phantom 440 may include information about the electrical characteristics and structure of the phantom 440. The information about the electromagnetic radiation electrode 430 may include information about the electrical characteristics and structure of the electromagnetic radiation electrode 430.

The analysis device 470 may generate reference data by applying an electromagnetic field analysis algorithm to the generated electromagnetic model (S520). The electromagnetic field analysis algorithm is an algorithm that calculates approximate solutions by applying numerical analysis techniques to the governing equations for electromagnetic fields. The governing equations are partial differential equations that represent the generation of electricity and magnetism, electric and magnetic fields, and the density of charges and currents. The numerical analysis techniques include the finite element method (FEM), the boundary element method (BEM), the method of moments (MOM), and the finite-difference time-domain (FDTD). The generated reference data may be stored in the analysis device 470. The reference data may be data about the electric field and power formed inside the phantom 440 when unit electric power is applied to the phantom 440.

The analysis device 470 may acquire measurement data (S530). The measurement data may be data on the electric field and power measured from the phantom 440. The analysis device 470 may acquire adjustment data by applying an electromagnetic field correction algorithm to the reference data and the measurement data (S540). The analysis device 470 may apply a linear electromagnetic field correction algorithm or a nonlinear electromagnetic field correction algorithm to the reference data and the measurement data. The analysis device 470 may generate adjustment data by correcting the measurement data based on the electric field pattern and power pattern inside the phantom 440.

The analysis device 470 may convert the generated adjustment data into quantitative values requested by a user. The analysis device 470 may graph the converted quantitative values and provide them to the user using a graphic user interface (GUI). The graph may be used to evaluate the performance of the electromagnetic radiation device.

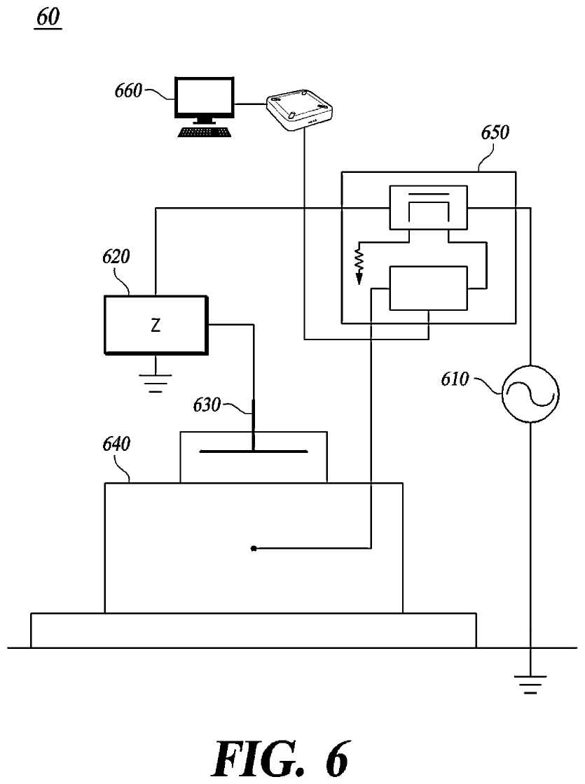

FIG. 6 is a structural diagram for explaining a system for monitoring the operating state of the electromagnetic radiation device, according to another embodiment of the present disclosure.

Referring to FIG. 6, the system for monitoring the operating state of the electromagnetic radiation device (hereinafter referred to as ‘monitoring system’) 60 includes a power source 610, an impedance matching circuit 620, an electromagnetic radiation electrode 630, a phantom 640, a detection unit 650, and an analysis device 660.

The power source 610 may generate power and transmit it to the electromagnetic radiation electrode 630 through the detection unit 650 and the impedance matching circuit 620. The electromagnetic radiation electrode 630 may irradiate the phantom 640 with electromagnetic waves using the power received from the power source 610.

The detection unit 650 may detect a power difference and a phase difference between a power signal of the power source 610 and a measurement signal received from the phantom 640. The power signal is a signal for the power generated from the power source 610 and the phase of the corresponding power. The measurement signal is a signal for the power measured from the phantom 640 and the phase of the corresponding power. The analysis device 660 may monitor the operating state of the electromagnetic radiation device in real time using the detected power difference and phase difference. The analysis device 660 may monitor the operating state of the electromagnetic radiation device in real time using initial state data and monitoring state data. In this case, the initial state data is data on the power difference and the phase difference between the power signal and the measurement signal detected by the detection unit 650 when the electromagnetic radiation device first operates. The monitoring state data is data on the phase change and the power change between the power signal and the measurement signal currently detected by the detection unit 650.

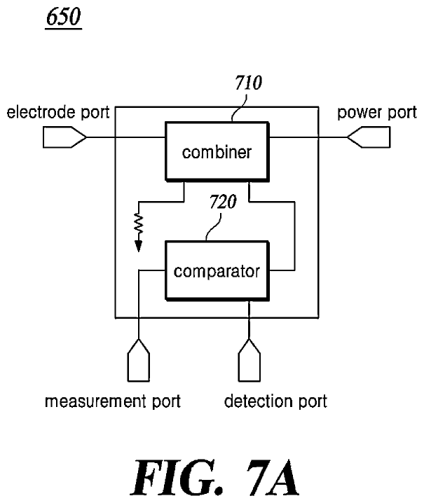

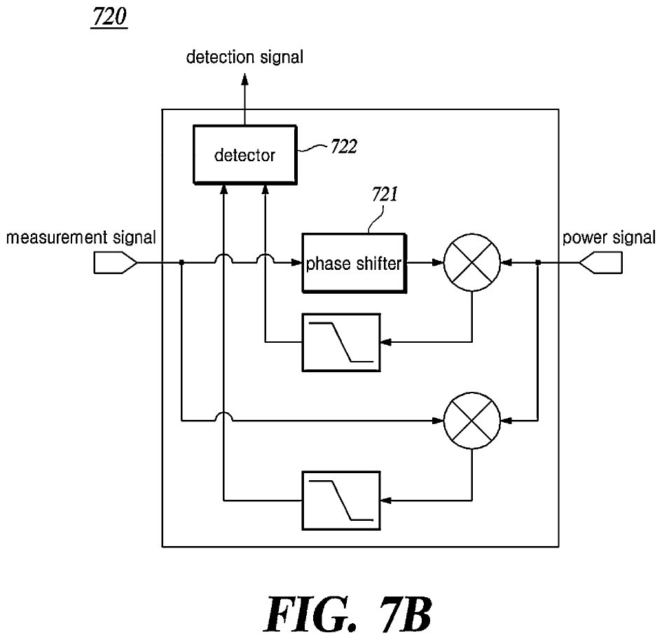

FIGS. 7A and 7B are structural diagrams for explaining the detection unit and a comparator included in the detection unit, according to one embodiment of the present disclosure.

Referring to FIG. 7A, the detection unit 650 includes a combiner 710, a comparator 720, an electrode port, a power port, a measurement port, and a detection port. The combiner 710 may receive a power signal from the power source 610 using the power port. The combiner 710 may transmit the power signal to the electromagnetic radiation electrode 630 using the electrode port. The combiner 710 may transmit the power signal to the comparator 720. The comparator 720 may receive a measurement signal from the electric field sensor inside the phantom 640 using the measurement port. The comparator 720 may generate a detection signal using the measurement signal and the power signal. The comparator 720 may transmit the detection signal to the analysis device 660 using the detection port. In this case, the detection signal is a signal for the phase change and the power change between the power signal and the measurement signal.

Referring to FIG. 7B, the comparator 720 includes a phase shifter 721 and a detector 722. The comparator 720 may receive a power signal from the combiner 710. The comparator 720 may receive a measurement signal from the electric field sensor inside the phantom 640. The phase shifter 721 may generate a phase-delayed measurement signal by delaying the phase of the measurement signal by 90 degrees. The detector 722 may receive a first signal combining the phase-delayed measurement signal and the power signal, and a second signal combining the measurement signal and the power signal. In this case, when the first signal and the second signal pass a low-pass filter, the first signal and the second signal may include a phase difference component and a magnitude difference component between the measurement signal and the power signal. The detector 722 may generate a detection signal using the first signal and the second signal. The detector 722 may transmit the detection signal to the analysis device 660.

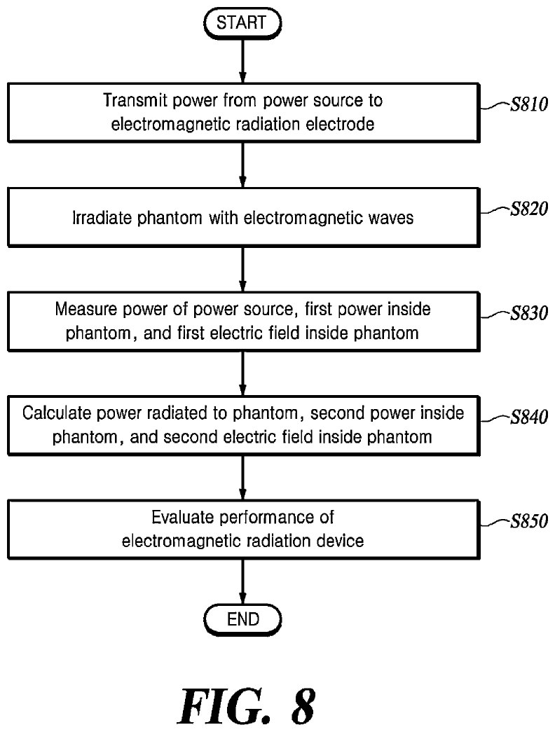

FIG. 8 is a flowchart for explaining a method of evaluating the performance of the electromagnetic radiation device, according to one embodiment of the present disclosure.

Referring to FIG. 8, the performance evaluation system 40 may transmit power from the power source 410 to the electromagnetic radiation electrode 430 (S810). The performance evaluation system 40 may irradiate the phantom 440 with electromagnetic waves using the power of the power source 410 (S820). The performance evaluation system 40 may measure the power of the power source 410, a first power inside the phantom 440, and a first electric field inside the phantom 440 (S830).

The performance evaluation system 40 may calculate the power radiated to the phantom 440, a second power inside the phantom 440, and a second electric field inside the phantom 440 (S840). The process of calculating the power radiated to the phantom 440, the second power inside the phantom 440, and the second electric field inside the phantom 440 may include calculating PLD using the conductivity of the phantom 440 and calculating a relative power ratio using the first power inside the phantom 440. The process of calculating the power radiated to the phantom 440, the second power inside the phantom 440, and the second electric field inside the phantom 440 may further include calculating the power radiated to the phantom 440 using the PLD and the relative power ratio.

The process of calculating the power radiated to the phantom 440, the second power inside the phantom 440, and the second electric field inside the phantom 440 may include generating an electromagnetic model using information about the phantom 440. The process of calculating the power radiated to the phantom 440, the second power inside the phantom 440, and the second electric field inside the phantom 440 may further include applying an electromagnetic field analysis algorithm to the electromagnetic model to calculate a third power inside the phantom 440 and a third electric field inside the phantom 440. The process of calculating the power radiated to the phantom 440, the second power inside the phantom 440, and the second electric field inside the phantom 440 may further include applying an electromagnetic field correction algorithm to the third power and the third electric field inside the phantom 440, and the first power and the first electric field inside the phantom 440 to calculate the second power and the second electric field inside the phantom 440.

The performance evaluation system 40 may evaluate the performance of the electromagnetic radiation device using at least one of the power radiated to the phantom 440, the second power inside the phantom 440, and the second electric field inside the phantom 440 (S850). The process of evaluating the performance of the electromagnetic radiation device may include graphing the second power inside the phantom 440 and the second electric field inside the phantom 440. The phantom 440 may include an electric field sensor for measuring the first power inside the phantom 440 and the first electric field inside the phantom 440 which is contained in the biological mimicry fluid. The phantom 440 may include a temperature sensor for measuring the temperature inside the phantom 440 which is contained in the biomimetic solid specimen.

Each element of the apparatus or method in accordance with the present invention may be implemented in hardware or software, or a combination of hardware and software. The functions of the respective elements may be implemented in software, and a microprocessor may be implemented to execute the software functions corresponding to the respective elements.

Various embodiments of systems and techniques described herein can be realized with digital electronic circuits, integrated circuits, field programmable gate arrays (FPGAs), application specific integrated circuits (ASICs), computer hardware, firmware, software, and/or combinations thereof. The various embodiments can include implementation with one or more computer programs that are executable on a programmable system. The programmable system includes at least one programmable processor, which may be a special purpose processor or a general purpose processor, coupled to receive and transmit data and instructions from and to a storage system, at least one input device, and at least one output device. Computer programs (also known as programs, software, software applications, or code) include instructions for a programmable processor and are stored in a “computer-readable recording medium.”

The computer-readable recording medium may include all types of storage devices on which computer-readable data can be stored. The computer-readable recording medium may be a non-volatile or non-transitory medium such as a read-only memory (ROM), a random access memory (RAM), a compact disc ROM (CD-ROM), magnetic tape, a floppy disk, or an optical data storage device. In addition, the computer-readable recording medium may further include a transitory medium such as a data transmission medium. Furthermore, the computer-readable recording medium may be distributed over computer systems connected through a network, and computer-readable program code can be stored and executed in a distributive manner.

Although operations are illustrated in the flowcharts/timing charts in this specification as being sequentially performed, this is merely an exemplary description of the technical idea of one embodiment of the present disclosure. In other words, those skilled in the art to which one embodiment of the present disclosure belongs may appreciate that various modifications and changes can be made without departing from essential features of an embodiment of the present disclosure, that is, the sequence illustrated in the flowcharts/timing charts can be changed and one or more operations of the operations can be performed in parallel. Thus, flowcharts/timing charts are not limited to the temporal order.

Although exemplary embodiments of the present disclosure have been described for illustrative purposes, those skilled in the art will appreciate that various modifications, additions, and substitutions are possible, without departing from the idea and scope of the claimed invention. Therefore, exemplary embodiments of the present disclosure have been described for the sake of brevity and clarity. The scope of the technical idea of the present embodiments is not limited by the illustrations. Accordingly, one of ordinary skill would understand that the scope of the claimed invention is not to be limited by the above explicitly described embodiments but by the claims and equivalents thereof.

What is claimed is:

1. A performance evaluation system comprising:
a power source for transmitting power to an electromagnetic radiation electrode;
an impedance matching circuit for matching an impedance of the power source and an impedance of the electromagnetic radiation electrode;
the electromagnetic radiation electrode for irradiating a phantom with electromagnetic waves using power of the power source;
the phantom that simulates a subject which is a target of electromagnetic wave irradiation;
an instrument for measuring the power of the power source, a first power inside the phantom, and a first electric field inside the phantom; and
an analysis device for calculating a power radiated to the phantom and evaluating performance of an electromagnetic radiation device using at least one of the power radiated to the phantom, a second power inside the phantom, and a second electric field inside the phantom.

2. The performance evaluation system of claim 1, wherein the phantom includes an electric field sensor, for measuring the first power inside the phantom and the first electric field inside the phantom, which is contained in a biological mimicry fluid.

3. The performance evaluation system of claim 2, wherein the phantom further includes a temperature sensor, for measuring a temperature inside the phantom, which is contained in a biomimetic solid specimen.

4. The performance evaluation system of claim 2, wherein the analysis device calculates a power loss density (PLD) using a conductivity of the phantom, calculates a relative power ratio using the first power inside the phantom, and calculates the power radiated to the phantom using the PLD and the relative power ratio.

5. The performance evaluation system of claim 2, wherein the analysis device generates an electromagnetic model using information about the phantom, and calculates a third power inside the phantom and a third electric field inside the phantom by applying an electromagnetic field analysis algorithm to the electromagnetic model.

6. The performance evaluation system of claim 5, wherein the analysis device calculates the second power inside the phantom and the second electric field inside the phantom by applying an electromagnetic field correction algorithm to the third power and the third electric field inside the phantom, and the first power and the first electric field inside the phantom.

7. The performance evaluation system of claim 6, wherein the analysis device graphs the second power inside the phantom and the second electric field inside the phantom.

8. A monitoring system comprising:
a power source for transmitting power to an electromagnetic radiation electrode;
an impedance matching circuit for matching an impedance of the power source and an impedance of the electromagnetic radiation electrode;
the electromagnetic radiation electrode for irradiating a phantom with electromagnetic waves using power of the power source;
the phantom that replaces a subject which is a target of electromagnetic wave irradiation;
a detection unit configured to detect a power difference between a power signal of the power source and a measurement signal inside the phantom and a phase difference between the power signal of the power source and the measurement signal inside the phantom; and
an analysis device for monitoring an operating state of an electromagnetic radiation device using at least one of the power difference and the phase difference.

9. The monitoring system of claim 8, wherein the phantom includes an electric field sensor, for measuring a power inside the phantom and an electric field inside the phantom, which is contained in a biological mimicry fluid.

10. The monitoring system of claim 9, wherein the phantom further includes a temperature sensor, for measuring a temperature inside the phantom, which is contained in a biomimetic solid specimen.

11. The monitoring system of claim 8, wherein the detection unit includes a combiner for transmitting the power signal to each of the electromagnetic radiation electrode and a comparator.

12. The monitoring system of claim 11, wherein the detection unit further includes the comparator for receiving the measurement signal inside the phantom and comparing the measurement signal and the power signal to detect the power difference and the phase difference.

13. The monitoring system of claim 8, wherein the power signal is a signal for the power of the power source and a phase of the power of the power source.

14. The monitoring system of claim 8, wherein the measurement signal is a signal for a power inside the phantom and a phase of the power inside the phantom.

15. A method performed by a performance evaluation system, the method comprising:
transmitting power of a power source to an electromagnetic radiation electrode;
irradiating a phantom with electromagnetic waves using the power of the power source;
measuring the power of the power source, a first power inside the phantom, and a first electric field inside the phantom;
calculating power radiated to the phantom, a second power inside the phantom, and a second electric field inside the phantom; and
evaluating performance of an electromagnetic radiation device using at least one of the power radiated to the phantom, the second power inside the phantom, and the second electric field inside the phantom.

16. The method of claim 15, wherein calculating the power radiated to the phantom, the second power inside the phantom, and the second electric field inside the phantom comprises:
calculating a power loss density (PLD) using a conductivity of the phantom and calculating a relative power ratio using the first power inside the phantom; and
calculating the power radiated to the phantom using the PLD and the relative power ratio.

17. The method of claim 15, wherein calculating the power radiated to the phantom, the second power inside the phantom, and the second electric field inside the phantom comprises:
generating an electromagnetic model using information about the phantom; and
applying an electromagnetic field analysis algorithm to the electromagnetic model to calculate a third power inside the phantom and a third electric field inside the phantom.

18. The method of claim 17, wherein calculating the power radiated to the phantom, the second power inside the phantom, and the second electric field inside the phantom further comprises:
applying an electromagnetic field correction algorithm to the third power and the third electric field inside the phantom, and the first power and the first electric field inside the phantom to calculate the second power inside the phantom and the second electric field inside the phantom.

19. The method of claim 15, wherein evaluating the performance of the electromagnetic radiation device comprises:
graphing the second power inside the phantom and the second electric field inside the phantom.

20. The method of claim 15, wherein the phantom includes:
an electric field sensor, for measuring the first power inside the phantom and the first electric field inside the phantom, which is contained in a biological mimicry fluid; and
a temperature sensor, for measuring a temperature inside the phantom, which is contained in a biomimetic solid specimen.
